(19) United States
(12) Patent Application Publication
(10) Pub. No.: US 2021/0057054 A1
(43) Pub. Date: Feb. 25, 2021

(54) METHOD FOR THE HARMONIZATION OF ASSAY RESULTS

(71) Applicant: Phadia AB, Uppsala (SE)

(72) Inventors: Per MATSSON, Uppsala (SE); Karl ANDERSSON, Vange (SE); Joseph BERNARDO, North Hampton, NH (US)

(21) Appl. No.: 15/733,402

(22) PCT Filed: Jan. 22, 2019
(86) PCT No.: PCT/US2019/014529

§ 371 Date: Jul. 21, 2020

Related U.S. Application Data
(60) Provisional application No. 62/620,114, filed on Jan. 22, 2018.

(51) Int. Cl. G16H 10/40; G01N 33/574; G16H 50/70

(52) U.S. Cl. CPC G16H 10/40; G16H 50/70; G01N 33/57434

(57) ABSTRACT

Methods for harmonization of test results from a biological sample in a multiplexed biochemical assay, wherein presence and/or concentration of multiple biomarkers are determined at the same time in the same sample, making test results obtained in different laboratories comparable comprise: quantifying a presence or concentration of at least two different biomarkers in a biological sample and in a harmonization standard sample, independently in each sample, by means of a defined multiplexed biochemical assay implemented in a defined type of analytical instrument; receiving the test results from the samples into a computer-based decision engine for harmonization of test results from biological samples; and transforming the test results received from the biological sample, which includes transforming the test results from the harmonization standard sample into generalized units and adjusting the test results of the biological sample into the generalized units, GE.

### FIELD OF THE INVENTION

The present invention relates generally to a method, assay device and kit for multiplexed biochemical assays, making test results obtained in different laboratories comparable. In particular, the present invention relates to the repeated use of different control samples that in concert allows for both harmonization of test results and diagnostics of the test equipment.

### BACKGROUND OF THE INVENTION

Biochemical measurements are commonly used to diagnose, monitor and guide treatment of diseases. While most biochemical assays provide single outputs, i.e. measure presence or concentration of one defined biomarker, the use of multiplexed assays is increasing. In a multiplexed assay, the presence or concentration of multiple biomarkers are determined at essentially the same time (e.g. same day or same week) in one or more aliquots of the same sample. Each biomarker may be analyzed using the same device or multiple devices may be required to analyze the entire set of biomarkers. Resulting data is then combined to form an output (for example a single risk estimate or a pattern of some sort indicative of sample characteristics). Multiplexed assays increase efficiency but complicate the calibration procedure.

A particular type of biochemical assay, the immunoassay, uses antibodies for the specific detection of presence or concentration of a protein or other antigen. Immunoassays can be competitive or noncompetitive, solid or liquid phase, and may or may not require a separation step. For detection purposes, immunoassays may use a labeled antigen or labeled antibody and one or more sites where a detectable reactions occurs.

The skilled person is well aware that sometimes the output from a biochemical assay is of arbitrary nature. It is possible to use the concentration of the biomarker as the output. It is however also possible to define the quantity of a biochemical in efficiency units, as is done for the Factor IX protein drug (manufactured by Baxter). Individuals with deficiency of this protein have hemophilia B, a coagulation disorder. Factor IX is quantified in arbitrary enzyme efficiency units, so that a defined quantity of the drug produces a defined enzyme activity, irrespective of the actual concentration of Factor IX. Among the biochemical assay there are multiple methods of output formats and standards. In general, the outputs have widely varying units, and varying linearity to response. This variability complicates evaluation of any biochemical output result. In cases where multiple assay results are to be combined, the situation grows even more complicated, and if different laboratories are implementing the same assay using different types of analytical instruments, the inherent lab-to-lab, instrument-to-instrument, and instrument type-to-instrument type variation will add to the overall variation.

The medical community has recognized that harmonization is required, and procedures have been implemented to control and standardize the quality of results of a given biochemical assay at a given laboratory. Many clinical/analytical laboratories implement assay verification at three levels. According to a first level, there is typically one or more calibration samples embedded in each assay, so as to produce known data to interpolate results for unknown samples. If the calibration curve does not meet predetermined standards, the assay results are deemed not reliable. According to a second level, there are locally produced positive control samples with known properties, which are independent from the calibration samples. If the calibration curve is not capable of reproducing the known properties of the positive control samples, the assay results are deemed not reliable. A third level, often referred to as “proficiency testing”, involves use of an external, trusted party to supply controls, and if the calibration curve is not capable of reproducing the known properties of the external control samples, the assay results are deemed not reliable.

One example of the third level control is the College of American Pathologists (CAP), an organization with the mission to foster and advocate for excellence in the practice of pathology and laboratory medicine worldwide (www.cap.org). CAP has an accreditation program which provides clinical laboratories access to external controls, so as to ensure that said laboratory is actually conducting clinical laboratory assays in a proper manner. For example, CAP requires that all laboratories that perform total serum IgE measurements demonstrate satisfactory performance in one of several masked external inter-laboratory proficiency testing surveys. Such proficiency testing surveys are conducted multiple times per year. In the extreme case, outlier clinical laboratories in the United States that fail a number of consecutive surveys may be subject to license revocation.

One example is described in the report “Proficiency Survey-Based Evaluation of Clinical Total and Allergen-Specific IgE Assay Performance” by Robert G Hamilton as published in Arch Pathol Lab Med. 2010;134:975-982, which is incorporated by reference herein. The program described in this publication relates to both total IgE determination and allergen specific IgE determination at approximately 200 certified clinical laboratories using equipment from multiple suppliers. The same set of samples was provided to all participating laboratories and analyzed centrally as described in the disclosure. It was noted that for total IgE, most assays had good performance although one technology provided systematically lower concentration results at high levels of IgE (FIG. 1 in “Proficiency Survey-Based Evaluation of Clinical Total and Allergen-Specific IgE Assay Performance”). For allergen specific IgE, the differences in concentration results between laboratories and technologies were much larger. For cat allergen, one technology reported an average concentration of IgE in a sample specimen exceeding 14 kU/L whereas two other technologies reported an average concentration of IgE in the same sample specimen of approximately 4 kU/L (FIG. 2 in “Proficiency Survey-Based Evaluation of Clinical Total and Allergen-Specific IgE Assay Performance”). This means that certified clinical laboratories can, for the very same sample, report concentration values of allergen specific IgE that differ more than a factor three. A common discrepancy between technologies is, according to the same disclosure, a 50% difference between the highest and the lowest reported concentration value (averaged over multiple clinically certified users of each technology) for the very same sample. It is important to note that such a difference between highest and lowest reported concentration values is deemed acceptable in the light of an available international standard from the World Health Organization (WHO), as evident in the disclosure “3rd International Standard for serum IgE Report of the international collaborative study to evaluate the candidate preparation” by Susan J Thorpe and co-authors, said disclosure being identified as WHO/BS/2013.2220. An international standard is typically provided for the purpose of standardizing the results from different laboratories.

A potential consequence of the acceptable variability between laboratories for IgE assays is the availability of extensive educational material such as “Serological IgE Analyses in the Diagnostic Algorithm for Allergic Disease.” by Hamilton and Oppenheimer as published in J Allergy Clin Immunol Pract. 2015 Nov.-Dec.; 3 (6):833-40. doi: 10.1016/j.jaip.2015, August 2016. Educational material of this kind prepares the user for interpretation of data that may differ from site to site, in lieu of a functional harmonization of assay results between laboratories.

Another example of a field where proficiency testing is employed is disclosed in “Standardization of Measurements for Cholesterol, Triglycerides, and Major Lipoproteins” by Warnick and co-authors as published in Lab medicine (August 2008, Volume 39, Number 8; DOI: 10.1309/6UL9RHJH1JFFU4PY). This publication discusses the standardization and proficiency testing of cholesterols and similar molecules. The disclosure shows that the proficiency testing for total cholesterol reported very good results where the participating laboratories were within the acceptance criteria in 97% of the test conducted. However, the determination of individual classes of cholesterol such as low-density lipoprotein (LDL) cholesterol and high-density lipoprotein (HDL) was more variable and only 84-90% of the tests were within acceptance criteria. This means that at least 1 out of 10 patient samples are at risk of being analyzed in an assay that is outside the recognized performance acceptance criteria for cholesterol determination.

Similar observations have been made for prostate cancer diagnosis and/or risk assessment. In the report “Between-method differences in prostate-specific antigen assays affect prostate cancer risk prediction by nomograms” by C Stephan and co-authors published in Clin. Chem. 2011 July; 57(7):995-1004. doi: 10.1373/clinchem.2010.151472, which is incorporated by reference herein, prostate cancer risk assessment using nomograms was evaluated using PSA values obtained from different platforms. This observation is of particular relevance when different instrument platforms are used to produce biomarker values in the same multiplexed assay.

The present invention is applicable to multiplexed assays irrespective of the number of assay devices used to obtain data. An example of a multiplexed assay where all data is produced by a single assay device is disclosed in the report “Validation of a multiplex chip-based assay for the detection of autoantibodies against citrullinated peptides.” by Hansson and co-authors as published in Arthritis Res Ther. 2012 Oct. 1; 14(5):R201, which is incorporated by reference herein. An example of a multiplexed assay where data is produced by different assay devices is disclosed in the patent application WO 2014079865, which is incorporated by reference herein.

The above described characteristics of single-analyte assays (i.e. assays that quantify the concentration of one component, such as PSA concentration in serum) are complicated but manageable. When combining multiple analytes into a multiplexed test, similar complications will occur for each analyte present in the multiplex panel, leading to considerable challenges in both production of multiplexed assays and interpretation of data from multiplexed assays. The present invention aims at solving such issues so as to improve the performance of multiplexed assays.

### SUMMARY OF THE INVENTION

According to one aspect, the present invention according to various embodiments improves the third level control for multiplexed assays through repeated use of external control samples, also denoted harmonization standards, and through embedding the harmonization standards in the assay interpretation.

According to an embodiment of the present invention, there is provided a method for harmonization of test results from biological samples in multiplexed biochemical assays, wherein a presence and/or concentration of each of a set of multiple biomarkers are determined at essentially the same time in the same sample. The method comprises quantifying a presence and/or concentration of at least two different biomarkers in a biological sample and in a harmonization standard sample, independently in each sample, by means of a defined multiplexed biochemical assay device implemented in a defined type of analytical instrument; receiving the test results from the samples into a computer-based decision engine for harmonization of test results from the biological sample; and transforming the test results from the biological sample using the computer-based decision engine. Transforming the test results from the biological sample includes transforming at least the test results from the harmonization standard sample into generalized units (GE). Finally, the transformed results of the harmonization standard sample are used to adjust the results of the biological sample into generalized units (GE).

The use of generalized units (GE) eliminates systematic error, improves the comparability of results between laboratories and technology platforms, and allows a centralized interpretation of results.

According to another embodiment of the present invention, there is provided a defined multiplexed biochemical assay device adapted to be implemented in a defined type of analytical instrument and containing a presence or concentration of at least two different biomarkers in a biological sample and in a harmonization standard sample, independently in each sample, that can be quantified by the analytical instrument.

According to an embodiment, the biochemical assay device comprises 10 or more independent and simultaneous assays, wherein a harmonization standard contains at least 90% of the biomarkers that are part of the assay. According to alternative embodiments, it may be preferable to continuously change the properties of the harmonization standard, so that multiple aspects of the multiplexed assay are tested regularly.

According to an embodiment, the biochemical assay device comprises a solid phase having immobilized thereon at least two different categories of ligands, wherein each category of ligands may be immobilized on a single component or on multiple components which collectively comprise the device, and:

the first category of the ligands binds specifically to a prostate cancer (PCa) biomarker, and includes a plurality of different ligands binding specifically to each of a plurality of different PCa biomarkers, preferably at least one of PSA, iPSA, tPSA, fPSA, and hK2, and optionally MSMB and/or MIC-1; and

the second category of the ligands binds specifically to a prostate cancer-related single nucleotide polymorphism (SNPpc), and includes a plurality of different ligands binding specifically to each of a plurality of different SNPpc, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

According to an embodiment, the solid phase further has a third category of ligand immobilized which binds specifically to a SNP-associated biomarker (SNPbm), and includes one or a plurality of different ligands binding specifically to one or each of a plurality of different SNPbm, such as at least one of rs1227732, rs3213764, rs1354774, rs2736098, rs401681, rs10788160, rs11067228, rs1363120, rs888663, and rs1054564.

According to another embodiment of the present invention, there is provided a kit comprising the biochemical assay device according to the above various embodiments described.

According to another embodiment of the present invention, there is provided a system for harmonization of test results from biological samples in multiplexed biochemical assays, wherein the system comprises a defined multiplexed biochemical assay device implemented in a defined type of analytical instrument, both adapted to quantify a presence or concentration of at least two different biomarkers in a biological sample and at least two different biomarkers in a harmonization standard sample, independently in each sample, a computer-based decision engine comprising a means for receiving the test results from the samples into a computer-based decision engine for harmonization of test results from the biological sample; and means for transforming the test results from the biological sample using the computer-based decision engine. The means for transforming the test results from the biological sample is arranged to transform the test results from the harmonization standard sample into generalized units and to adjust the results of the biological sample into generalized units.

According to a second aspect of the invention, there is provided a computer program product comprising instructions for causing a computer executing the instructions to perform the parts of the method provided by the decision engine.

In one embodiment, the computer-based decision engine is included in the system.

In one embodiment, all components of the computer-based decision engine are provided by the computer program product integrated in a unitary device, which may be a server, a personal computer, an analytical instrument, or any other device with data processing ability.

According to an aspect, harmonization standards can be linked to manufactured standards.

According to an aspect, a supplier of a new batch of harmonization standards can be required to perform the harmonization as described above before delivery to customer.

An advantage with various embodiments of the present invention is that they facilitate comparisons between test results obtained in different laboratories.

### BRIEF DESCRIPTION OF THE DRAWINGS

Embodiments of the present invention will now be described with reference to drawing figures, of which:

FIG. 1 shows a flowchart of a method according to an embodiment of the invention.

FIG. 2 shows a defined multiplexed biochemical assay device according to an embodiment of the disclosure.

FIG. 3 shows a block schematic of a system according to an embodiment of the invention.

FIG. 4 shows a block schematic of a computer-based decision engine.

FIG. 5 shows the average total PSA value for harmonization standards (in arbitrary units) on the y-axis and the median unknown sample (i.e. patient sample) total PSA value on the same substrate on the x-axis (in the same arbitrary units).

FIG. 6 shows the average MSMB value for harmonization standards (in arbitrary units) on the y-axis and the median unknown sample (i.e. patient sample) MSMB value on the same substrate on the x-axis (in the same arbitrary units).

FIG. 7 shows the average GDF-15 value for harmonization standards (in arbitrary units) on the y-axis and the median unknown sample (i.e. patient sample) GDF-15 value on the same substrate on the x-axis (in the same arbitrary units).

### DETAILED DESCRIPTION OF EMBODIMENTS

For the purpose of this disclosure and for clarity, the following definitions are made:

The term “diagnostic assay” refers to the detection of the presence or characterization of a pathologic condition. It may be used interchangeably with “diagnostic method” or “diagnostic test.” Diagnostic assays may differ in their sensitivity and specificity.

The term “prognostic assay” refers to the assessment of risk of developing a pathologic condition. It may be used interchangeably with “prognostic method” or “prognostic test.” Prognostic assays are, when providing a prognosis on if a particular event will occur, similar to diagnostic assays and may in such cases differ in their sensitivity and specificity. One such example is the prognostic assay forecasting if active therapy is required.

The term “assay device” refers to a physical device for performing an assay which may comprise one or more components, collectively comprising the device.

One measure of the usefulness of a diagnostic tool is “area under the receiver—operator characteristic curve”, which is commonly known as ROC-AUC statistics. This widely accepted measure takes into account both the sensitivity and specificity of the tool. The ROC-AUC measure typically ranges from 0.5 to 1.0, where a value of 0.5 indicates the tool has no diagnostic value and a value of 1.0 indicates the tool has 100% sensitivity and 100% specificity.

The term “sensitivity” refers to the proportion of all subjects requiring active treatment that are correctly identified as such (which is equal to the number of true positives divided by the sum of the number of true positives and false negatives).

The term “specificity” refers to the proportion of all subjects not requiring active treatment (i.e. suitable for watchful waiting) that are correctly identified as such (which is equal to the number of true negatives divided by the sum of the number of true negatives and false positives).

The term “analyte” refers to a target biochemical/biomarker that is subject to detection and/or quantification in an assay. Examples of analytes are proteins, oligonucleotides or chemical compounds.

The term “recognition element” refers to an entity capable of interacting with a specific analyte. One non-limiting example of a recognition element is an antibody which specifically binds a defined analyte. Another non-limiting example of a recognition element is an aptamer which specifically binds a defined analyte.

The term “biomarker” refers to a protein, a part of a protein, a peptide, a polypeptide, an oligonucleotide (DNA or RNA), a chemical compound, metabolites, catabolites, or circulating cells (such as circulating tumor cells to mention one non-limiting example) which may be used as a biological marker, e.g. for diagnostic purposes. In an assay, a biomarker is typically the analyte.

The term “harmonization standard” refers to a sample having properties known to the manufacturer but typically unknown to user. A harmonization standard covers at least 50% of the individual assays or biomarkers that are analyzed in a multiplexed assay.

The term “multiplexed biochemical assay” refers to an assay which combines at least two different biomarker values (such as concentration of biomarker in a biological sample or detectable presence of biomarker in a biological sample, as two non-limiting examples) for the purpose of assessing the condition of the donor of the biological sample (such as estimating risk for a cancer disease, to mention a non-limiting example). The biomarker values may be obtained using multiple assay components, each producing at least one biomarker value and collectively comprising the multiplexed assay, or may be obtained using only one assay device producing all desired biomarker values, or any other combination thereof that in concert produces all desired biomarker values. A multiplexed biochemical assay in the context of the present disclosure produces all desired biomarker values within a time-frame that is similar to the longest storage time of the biological sample. In the case of a blood sample which is processed into plasma or serum, the typical longest storage time in a refrigerator is about 2-4 weeks for protein biomarker assays, because when stored longer the degradation of the sample as such may have an impact on the accuracy of the determined biomarker values. In specific embodiments, multiplexed biochemical assay combines at least ten different biomarker values, at least twenty different biomarker values, at least thirty different biomarker values, at least forty different biomarker values, or at least fifty different biomarker values.

The Single Nucleotide Polymorphism Database (dbSNP) is an archive for genetic variation within and across different species developed and hosted by the National Center for Biotechnology Information (NCBI) in collaboration with the National Human Genome Research Institute (NHGRI), both located in the US. Although the name of the database implies a collection of one class of polymorphisms only (i.e., single nucleotide polymorphisms (SNP)), it in fact contains a range of molecular variation. Every unique submitted SNP record receives a reference SNP ID number (“rs#”; “refSNP cluster”). In the present application, SNP are mainly identified using rs# numbers.

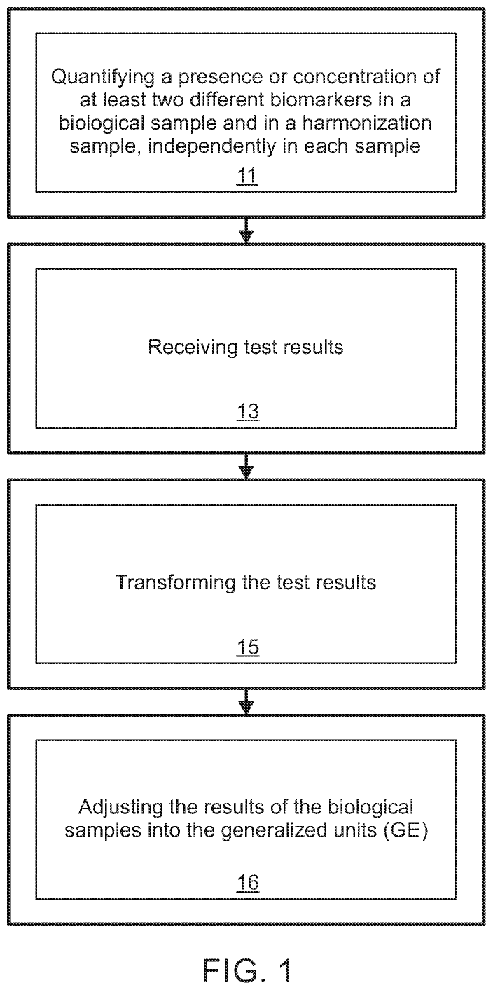

Reference is now made to FIG. 1, which shows a flowchart of a method according to an embodiment of the disclosure.

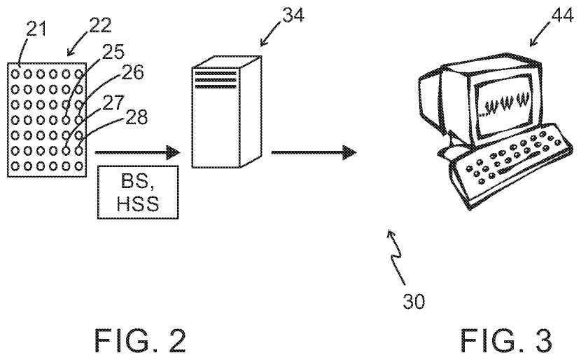

In a first step a), a presence or concentration of at least two different biomarkers in a biological sample BS and in a harmonization standard sample HSS, independently in each sample BS, HSS is quantified 11, by means of a defined multiplexed biochemical assay device implemented in a defined type of analytical instrument, which both are described below in more detail with reference to FIGS. 2 and 3.

Then, in a second step b), the test results Test resultₛₐₘₚₗₑ, Test result_{harmonization} from the samples BS, HSS are received 13 into a computer-based calculation engine 44 for transforming 15, step c), test results Test resultₛₐₘₚₗₑ received from the sample BS, and harmonization of test results Test resultₛₐₘₚₗₑ from the biological sample BS.

Transforming 15 the test results Test resultₛₐₘₚₗₑ from the biological sample BS includes transforming at least the test results Test result—harmonization from the harmonization standard sample HSS into generalized units (GE). Finally, the test results Test resultₛₐₘₚₗₑ of the biological sample BS are adjusted into generalized units (GE).

According to an embodiment, the steps a) to c) are repeated at least twice within a particular time frame.

According to an embodiment, the steps a) to c) are repeated with a different biological sample and using the results Test result_{harmonization} from the at least two harmonization standards HSS quantified at different time points as input in the transformation 15 into generalized units (GE).

According to an embodiment, the steps a) to c) are repeated with a different biological sample BS and a different harmonization standard HSS, and using the results Test result_{harmonization} from the at least two harmonization standards HSS quantified at different time points as input in the transformation 15 into generalized units (GE).

A non-limiting example is described. A biological sample is assayed to measure biomarkers lto n in the sample, with the measured amounts of the biomarkers from the sample designated as SM1 to SMn, where n can be an integer of 2 to 10, 20, 30, 40, 50, or more. A harmonization sample with the biomarkers 1 to n (or more or less biomarkers) is assayed under the same conditions (i.e., same laboratory, equipment and operator), with the measured amounts of the biomarkers designated as HM1 to HMn. Using a computer-based decision engine, the measured amounts of the biomarkers in the harmonization sample are transformed to generalized units by comparison with known generalized units KGE1 to KGEn for the harmonization sample biomarkers and generation of a function/algorithm based on the differences between all measured harmonization biomarkers HM1 to HMn and the known generalized units KGE1 to KGEn. That function/algorithm is then used to transform SM1 to SMn to generalized units for the sample biomarkers, SGE1 to SGEn, which can then be used for diagnostic evaluation.

Harmonization is used to adjust for technology-to-technology differences, laboratory-to-laboratory differences and disposable-to-disposable differences. If both HM1 and HM2 for a particular laboratory have values as expected (manufacturer-provided true concentration of the markers in each harmonization standard), then only technology-to-technology correction will be applied. For example, certain PSA (Prostate-specific antigen) platforms are known to have systematic differences of 5-10% reduction. So if HM1 and HM2 are measured on such a device, the HM1 and HM2 as measured will be expected to be 10% lower than the known generalized units and consequently all samples will need adjustment by multiplication of 1.1 to match the measurement profile of the data used for creating the algorithm. Now, should HM1, HMn sporadically deviate from expected values, there might be a disposable issue, i.e., some disposables significantly over or under estimate concentrations. If all HM1, HM2, . . . HMn are consistently different from expected values, one can assume that also the sample data is consistently different in the same manner, and HM1, HM2, . . . HMn values can guide the transformation of sample data. For example, if HM1=0.5*expected value KGE1 and HM2=0.5*expected value KGE2, then all samples are multiplied by 2 to produce values in generalized units. Thus, in a simplified view, (Generalized unit sample value)=(measured sample value)/AVERAGE (HM1/KGE1; HM2/KGE2; HMn/KGEn). Therefore, if HM1=2*KGE1 and same for all other HMn/KGEn values, then average=2 and measured samples would be divided by 2 to adjust for unexpectedly high HM1, HM2, . . . HMn. As will be apparent, in a multiplex system, the transformation step as described is considerably complex.

KGE1 to KGEn and HM1 to HMn which have small differences (less than a predetermined minimum), may indicate noise in the measurement system and for differences less than a predetermined minimum (reflecting the cumulative noise), the computer-based decision engine may be programed to not apply corrections, i.e., the system is noise limited. On the other hand, for KGE1 to KGEn and HM1 to HMn with large differences, the computer-based decision engine may be programed to warn the user when large differences (for example, greater than a predetermined maximum) are noted, and one of several warnings can be made. For example, stochastic/random and/or large variations of HM/KGE ratios may be indicative of sloppy operators, poor and/or varying disposable quality, etc. while systematic and consistent deviations may be indicative that something is systematically done different in the laboratory at hand, and may require an update of the KGE values for that particular laboratory. HM/KGE ratios may also have seasonal variation, such as the assay performance during winter being different than the assay performance during summer as a non-limiting example.

According to an embodiment, the steps a) to c) are repeated with a different biological sample (BS) or a different harmonization standard (HSS), and using the results (Test result_{harmonization}) from the at least two harmonization (HSS) standards quantified at different time points as input in the transformation (15) into generalized units (GE).

Now reference is made to FIG. 2.

FIG. 2 shows an embodiment of a defined multiplexed biochemical assay device 22 adapted to be implemented in a defined type of analytical instrument 34 and having a presence or concentration of at least two different biomarkers 25, 26 in a biological sample BS and two different biomarkers 27, 28 in a harmonization standard sample HSS. One non-limiting example of a multiplexed biochemical assay device 22 is a microarray. The microarray 22 typically comprises a solid support 21 onto which a plurality of recognition elements 25, 26, 27, 28, each being capable of specifically interacting with and immobilizing different defined analytes. Commonly, antibodies are used as recognition elements. After the analytes have bound to their respective recognition elements, the solid support is washed and a detection reagent is put in contact with the solid support. A detection reagent is typically a labeled molecule binding to one or more analytes, making presence of analyte detectable through the label. When performing a microarray assay, the typical workflow comprises contacting the solid support with a biological sample followed by incubation 10-60 minutes. Thereafter, the solid support 21 is washed with a liquid devoid of all target analytes. A labeled detection reagent (for example, a reagent labeled with a fluorescent moiety) is added and incubated for 10-60 minutes. After a final wash of the solid support 21, the microarray 22 can be scanned in a fluorescence scanner to reveal which spots on the microarray 22 demonstrated presence of analyte.

Then, the analytes are subject to quantization in the analytical instrument 34, corresponding to step a) described above in relation to FIG. 1.

According to an embodiment, the biochemical assay device comprises one or more solid phase components 21 having immobilized thereon at least two different categories of ligands, wherein:

the first category of the ligands binds specifically to a PCa biomarker, and includes a plurality of different ligands binding specifically to each of a plurality of different PCa biomarkers, preferably at least one of PSA, iPSA, tPSA, fPSA, and hK2, and optionally MSMB and/or MIC-1; and

the second category of the ligands binds specifically to a SNPpc, and includes a plurality of different ligands binding specifically to each of a plurality of different SNPpc, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

According to an embodiment, the solid phase 21 further has a third category of ligand immobilized which binds specifically to a SNPbm, and includes one or a plurality of different ligands binding specifically to one or each of a plurality of different SNPbm, such as at least one of rs1227732, rs3213764, rs1354774, rs2736098, rs401681 , rs10788160, rs11067228, rs1363120, rs888663, and rs1054564.

Now is referred to FIG. 3, which shows a system 30 for harmonization of test results from biological samples in multiplexed biochemical assay devices 22 such as microarrays, and implementing the method described above described above in relation to the embodiment described and shown in FIG. 1.

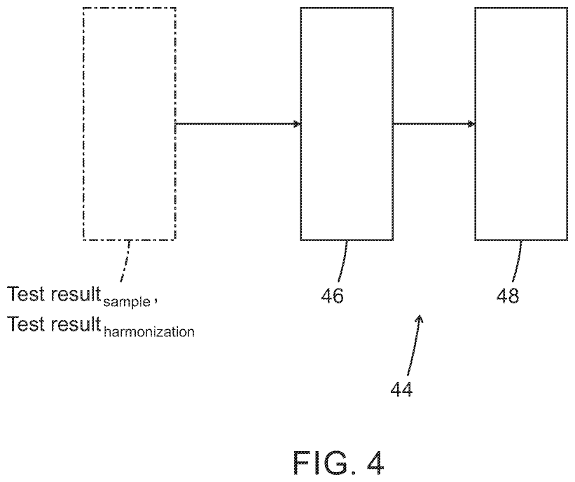

The system 30 comprises a defined multiplexed biochemical assay device 22 implemented in a defined type of analytical instrument 34. The biochemical assay device 22 and analytical instrument 34 are both, typically, together adapted to perform step a), i.e. to quantify 11, a presence or concentration of at least two different biomarkers in a biological sample and a harmonization standard sample, independently in each sample. The system 30 further comprises a computer-based decision engine 44, comprising a component 46 for receiving 13 the test results from the samples into the computer-based decision engine 44; and a component 48 for transforming 15 the test results received from the biological sample. These components 46, 48 are schematically indicated in FIG. 4. The component 48 for transforming 15 the test results from the biological sample is arranged to transform the test results from the harmonization standard sample into generalized units (GE) and to adjust the unknown results of the biological sample into generalized units (GE).

According to an aspect of the disclosure, the components 46 and 48 that define the decision engine 44 in this example may be implemented by special-purpose software (or firmware) run on one or more general-purpose or special-purpose computing devices. Such a computing device may include one or more processing units, e.g. a CPU (“Central Processing Unit”), a DSP (“Digital Signal Processor”), an ASIC (“Application-Specific Integrated Circuit”), discrete analogue and/or digital components, or some other programmable logical device, such as an FPGA (“Field Programmable Gate Array”). In this context, it is to be understood that each “component” of the decision engine 44 refers to a conceptual equivalent of a method step; there is not always a one-to-one correspondence between components and particular pieces of hardware or software routines. One piece of hardware sometimes comprises different components. For example, the processing unit may serve as one component when executing one instruction, but serve as another component when executing another instruction. In addition, one component may be implemented by one instruction in some cases, but by a plurality of instructions in some other cases. The computing device may further include a system memory and a system bus that couples various system components including the system memory to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAM) and flash memory. The special-purpose software may be stored in the system memory, or on other removable/non-removable volatile/non-volatile computer storage media which is included in or accessible to the computing device, such as magnetic media, optical media, flash memory cards, digital tape, solid state RAM, solid state ROM, etc. The computing device may include one or more communication interfaces, such as a serial interface, a parallel interface, a USB interface, a wireless interface, a network adapter, etc. One or more I/O devices may be connected to the computing device, via a communication interface, including e.g. a keyboard, a mouse, a touch screen, a display, a printer, a disk drive, etc. The special-purpose software may be provided to the computing device on any suitable computer-readable medium, including a record medium, a read-only memory, or an electrical carrier signal.

In an embodiment of the method, the measurement of a presence or absence of a SNP (belonging to any category of SNPs) comprises measuring the number of alleles of said SNP. In an embodiment, one or two alleles corresponds to a presence of said SNP and zero alleles corresponds to an absence of said SNP in said individual; wherein zero alleles corresponds to homozygous negative for said SNP, one allele corresponds to heterozygous positive, and two alleles corresponds to homozygous positive. Suitable categories of SNPs include, but are not limited to, SNPs related to the disease that the diagnostic or prognostic assay is related to, SNPs related to risk factors for the disease that the diagnostic or prognostic assay is related to. Non-limiting examples of risk factors are protein biomarker levels and obesity.

In an embodiment, the above described method step a) comprises using one or more analytical devices including, without limitation, an ELISA assay device, a microarray assay device, an immunoprecipitation assay device, an immunofluorescence assay device, a radio-immuno-assay device, or a mass spectrometry device using matrix-assisted laser desorption/ionization (MALDI), for the measurement of a presence or concentration of a PCa biomarker.

In an embodiment, which may be combined with the above-mentioned embodiment, the above-described method comprises using a mass spectrometry device using matrix-assisted laser desorption/ionization (MALDI), for the measurement of a presence or absence of a SNP.

In an embodiment, the above described analytical instrument comprises one or more analytical devices including, without limitation, an ELISA assay device, a microarray assay device, an immunoprecipitation assay device, an immunofluorescence assay device, a radio-immuno-assay device, or a mass spectrometry device using matrix-assisted laser desorption/ionization (MALDI), for the measurement of a presence or concentration of a PCa biomarker. In an embodiment, which may be combined with the above-mentioned embodiment, the above-described assay device comprises a mass spectrometry device using matrix-assisted laser desorption/ionization (MALDI), for the measurement of a presence or absence of a SNP.

According to a further aspect of the invention, a test kit is provided for performing step a) measuring a presence or concentration of at least two biomarkers and optionally measuring a presence or absence of at least one SNP of the above-described method for indicating a presence or non-presence of a defined disease or condition in an individual, comprising a corresponding assay device as described above and use of at least one harmonization standard sample HSS to make it possible to transform the measured values into generalized units (GE), and finally estimating the likelihood of an individual having said disease or condition.

One potential application for the present method is a diagnostic assay for determining presence or non-presence of prostate cancer, wherein suitable biomarkers include, but are not limited to, PSA, iPSA, tPSA, fPSA, and hK2, and optionally MSMB and/or MIC-1 ; and wherein suitable SNP include, but are not limited to, rs582598, rs439378, rs2207790, rs1046011, rs10458360, rs7525167, rs10489871, rs7529518, rs4245739, rs4512641, rs10178804, rs11900952, rs1873555, rs10191478, rs6755901, rs6545962, rs721048, rs2710647, rs12612891, rs2028900, rs1009, rs12233245, rs6760417, rs10496470, rs10199796, rs12475433, rs16860513, rs12151618, rs3765065, rs13017302, rs12988652, rs871688, rs749264, rs3771570, rs4346531, rs6770955, rs12637074, rs2660753, rs13319878, rs6437715, rs2162185, rs1515542, rs2270785, rs9830294, rs1439024, rs6762443, rs888507, rs6794467, rs12490248, rs1477886, rs4833103, rs3796547, rs17779822, rs2366711, rs16849146, rs1894292, rs12640320, rs3805284, rs12500426, rs4699312, rs17021918, rs7679673, rs2047408, rs2647262, rs12506850, rs7658048, rs2078277, rs12505546, rs13113975, rs4246742, rs2736098, rs401681, rs11134144, rs10060513, rs40485, rs2087724, rs1482679, rs16901841, rs1295683, rs2070874, rs7752029, rs2018334, rs9358913, rs1140809, rs409558, rs3096702, rs9267911, rs2025645, rs9359428, rs6569371, rs2813522, rs1933488, rs712242, rs6934898, rs9456490, rs651164, rs3120137, rs9364554, rs9457937, rs10486562, rs10807843, rs7801918, rs6962297, rs2465796, rs6957416, rs7777631, rs2272316, rs6961773, rs2132276, rs13265330, rs16887736, rs2911756, rs2272668, rs2339654, rs1380862, rs9297746, rs12543663, rs10086908, rs16901922, rs1016343, rs17832285, rs16901979, rs4871779, rs10107982, rs16902094, rs620861, rs17467139, rs6983267, rs9297756, rs10094059, rs7818556, rs1992833, rs986472, rs12552397, rs4273907, rs4237185, rs753032, rs11253002, rs2386841, rs10795841, rs10508422, rs7075945, rs10508678, rs539357, rs10826398, rs3818714, rs7090755, rs10993994, rs4382847, rs1891158, rs10887926, rs10788160, rs6579002, rs10832514, rs7358335, rs1944047, rs3019779, rs10896437, rs12793759, rs7106762, rs7102758, rs2449600, rs585197, rs2509867, rs11568818, rs7125415, rs11601037, rs11222496, rs4570588, rs6489721, rs3213764, rs17395631, rs4423250, rs11168936, rs10875943, rs3759129, rs902774, rs1827611, rs4760442, rs11610799, rs6539333, rs11067228, rs7485441, rs6489794, rs4119478, rs17070292, rs2293710, rs17256058, rs1950198, rs2331780, rs7141529, rs12880777, rs17123359, rs785437, rs524908, rs12903579, rs7178085, rs7164364, rs896615, rs11634741, rs9972541, rs12594014, rs11631109, rs1558902, rs8044335, rs2738571, rs885479, rs385894, rs684232, rs4925094, rs17138478, rs11649743, rs2107131, rs7213769, rs12946864, rs306801, rs138213197, rs1863610, rs17224342, rs9911515, rs12947919, rs966304, rs17744022, rs7234917, rs1943821, rs2227270, rs1363120, rs888663, rs122773222, rs1054564, rs4806120, rs11672691, rs758643, rs3745233, rs6509345, rs2659051, rs2735839, rs1354774, rs2691274, rs6090461, rs2297434, rs6062509, rs2315654, rs2823118, rs2838053, rs398146, rs16988279, rs2269640, rs4822763, rs132774, rs747745, rs5978944, rs6530238, rs5934705, rs5935063, rs4830488, rs17318620, rs5945619, rs5945637, rs11091768, rs2473057, rs5918762, rs4844228, rs6625760 and rs17324573.

The present invention according to various embodiments described above provides harmonization of assay results obtained from different laboratories where potentially different instrument platforms have been used. The harmonization is achieved by transforming assay values into generalized units which are comparable across instrument platforms. Essential for the method to work is the use of a harmonization kit, which is designed so that the properties of substantially all target analytes in a multiplexed biochemical assay device are verified with one or a few harmonization standards. To handle any deviations from expected performance, each result produced in one assay is transformed into generalized units (GE) where multiple aspects are considered, including, but not limited to, systematic deviation in the assay performance as quantified through the harmonization control sample in the present assay, any systematic drift within the laboratory or the instrumentation as quantified using the current harmonization standard result in combination with multiple previous harmonization standard results, and the performance profile of the instrument type used for obtaining the assay results.

A basic principle of the invention, according to an aspect, can be described in the following manner. The inventive harmonization method disclosed and claimed according to various embodiments aims at harmonizing assay output from different laboratories which potentially use different types of analytical equipment (instrument platforms). To achieve harmonization, each set of unknown samples (typically patient samples) are complemented with a harmonization standard. Since it is known that different laboratories and technologies may be systematically different in a manner that changes over time, the results of the harmonization standard is used to adjust the results of all unknown samples into generalized units. The use of generalized units eliminates the sum of systematic error and improves the comparability of results between laboratories and technology platforms and allows a centralized interpretation of results. Therefore, all diagnostic or prognostic or other clinical decisions are made using generalized units, so as to achieve the same performance of the diagnostic/prognostic method irrespective of laboratory or instrument type.

The harmonization method requires that both the laboratory and the equipment as such are characterized in order to retrieve the relationship between infrastructure/hardware and the generalized units (GE).

An analytical instrument is characterized in a manner that reveals how output from that instrument type is transformed to comply with generalized units.

A laboratory which is using a previously characterized analytical instrument for the purpose of conducting a generalizable assay must also be characterized to reveal how the impact of said laboratory infrastructure and standard operating procedures on assay output shall be transformed to comply with generalized units.

An analytical instrument of a type that has been previously characterized need typically not be characterized if a different laboratory acquires such an instrument. Each new installation of (any) instrument in a laboratory typically requires a repeated laboratory characterization for the harmonization of output from said newly installed instrument.

Once the analytical instrument and the laboratory are characterized to produce assay output in generalized units, the laboratory uses the analytical instrument for defined assays wherein each assay typically includes one or more harmonization standards. A harmonization standard is designed to serve both as an independent control (a third level control) for the majority of the individual assays in the multiplexed system, and as a reference point for assay result generalization. At the completion of the assay, assay output (preferably raw data output) is typically transferred to a central (digital) facility wherein the actual assay results are transformed to generalized units. Factors that have an impact on the transformation include (but are not limited to) harmonization standard results from the present assay, harmonization standard result from other laboratories where the same harmonization standard batch was used, instrument characteristics, laboratory characteristics, batch of reagents used for operation and calibration of the assay, and the temporal evolvement of harmonization standard results at the present laboratory.

The harmonization standard need not be composed in the same manner all the time. It is in fact preferable to continuously change the properties of the harmonization standard so that multiple aspects of the multiplexed assay are tested regularly. For example, if an assay measures the concentration of four biomarkers in blood, there could be reasonable to have two different harmonization standards: one where two biomarkers are present at high concentration and the other two at low concentration, and a second harmonization standard with the opposite concentration profile. By systematically and regularly (typically daily or weekly) switching harmonization standard in each assay, not only the function of the assay will be quantified in each assay through the test of measuring the known high-high-low-low (or vice versa) concentration profile, but after having assayed both harmonization standards, the dynamic range of the assay is verified because one known low concentration and one known high concentration for each of the included biomarkers are tested within a short timeframe.

Harmonization standards may further be tailored to investigate particular aspects of a multiplexed assay. For example, if unintentional cross reactivity is suspected in a new batch of reagents, a set of harmonization standards can be designed to investigate if the suspected issue was present or not. In a similar manner, the same batch of harmonization standards can be distributed to multiple different laboratories which make it possible to compare and correct for systematic small laboratory-to-laboratory deviations, as well as identifying if any particular laboratory is experiencing problems with a particular assay.

Multicomponent biochemical assay devices are impractical to calibrate and control one-by-one. Hence regular control samples and external standards are neither straightforward to implement, nor to evaluate. With the use of a harmonization standard, a multiplexed control becomes available and complements any other attempts to control the assay. In cases where the known harmonization standard result profile is kept confidential, it also serves as a security or authenticity control. A reported result profile for a defined and uniquely identified harmonization standard which differs significantly from the known and expected result profile indicates that either technical errors have occurred during the processing of the harmonization standard, or the harmonization standard used by the laboratory is counterfeit. Counterfeit reagents may jeopardize analytical accuracy and hence constitute a major risk for the patient.

Multicomponent biochemical assay devices are difficult to produce in a manner that all biomarkers are assayed at the same precision and the same dynamic range in irrespective of production batch. Under the assumption that one assay has a production yield of 95% (i.e. 19 out of 20 production attempts are successful), a multicomponent biochemical assay device comprising 10 individual assays located on the same solid support will have a production yield of (0.95 \hat{ }10)*100%=59.8%. According to our best understanding, this fact is a bottleneck in the development of multicomponent assays where protein molecules are incorporated, because proteins are sensitive and difficult to use in production, which automatically leads to poor production yield in multicomponent assays. The present invention makes it possible to accept lower performance of the multicomponent assay as such because the harmonization standard is used to adjust for deviations detected on the solid support when evaluating unknown samples. As a non-limiting example, assume that the implementation of generalized units through harmonization procedure make it possible to reduce performance requirements on each individual assay, hence accepting weaker assay performance and correcting the output through use of the harmonization standard. In cases where the initial production yield is 95% and the implementation of a harmonization procedure increases the yield to 98%, the overall production yield of a multicomponent assay comprising 10 individual assays located on the same solid support will be (0.98 \hat{ }10)*100%=81.7%. This means that also a small increase in production yield for individual assays can have a major impact on over-all production yield for multicomponent assays. The use of a harmonization standard is an external method for increasing production yield and will not require any changes to existing production methods.

In cases where the assay is conducted on a solid support comprising a multitude of independent spots, each spot having a recognition element such as an antibody attached, harmonization aspects can also be embedded in the solid support of the assay. For example, it is possible to attach the same recognition element on multiple spots, where a subset of the spots have a known lower density of said recognition element and others have known higher density of said recognition element. When assayed using a harmonization standard it is then possible to assess spot production variation (by comparing results from multiple spots of the same density) and spot production linearity (by comparing results from multiple spots of different density). Variation of spot production linearity may result in variation of assay dynamic range. By monitoring spot production linearity in situ through a harmonization standard, it becomes possible to adjust results to compensate for altered assay characteristics such as dynamic range.

It is further possible to include a completely irrelevant molecule in the harmonization standard and a corresponding multicomponent assay. For example, if a test is designed for use in humans, the harmonization standard can be supplemented with a mouse protein and the solid support could be designed to include assays for both the intended human biomarkers but also for the supplemented mouse protein. Such an artificial assay embedded in the multicomponent assay and the harmonization standard is beneficial because it serves as a functional control and a steady point throughout assay and evaluation. Such a steady point is beneficial for assessing external damage to components, such as transportation of reagents or solid supports at elevated temperatures that may reduce or even destroy performance of the assay, which may constitute a major risk for the patient.

Even though this invention is primarily applicable to an assay that estimates the concentration of multiple biomarkers, certain embodiments are applicable also to qualitative assays such as genotyping assay. As a non-limiting example, a harmonization standard as applied in a genotyping assay wherein a multitude of single nucleotide polymorphisms (SNP) are measured has the ability to serve as an authenticity control: due to the known genotyping profile of the harmonization standard, the measured genotype can be matched to the known genotype so as to confirm assay function and authentic assay. In a genotyping situation, it is further possible to embed irrelevant molecules or designing assays for irrelevant SNPs so as to serve as a functional control, similar to as previously discussed.

In order to illustrate how important suppression of systematic error can be, results from a large population based study of prostate cancer screening were reviewed. In this study, multiple protein biomarkers, multiple SNP and multiple other information-related factors (such as age and family history of prostate cancer, etc.) were combined into a risk score which was proven highly functional for the purpose of detecting prostate cancer as disclosed in “Prostate cancer screening in men aged 50-69 years (STHLM3): a prospective population-based diagnostic study” by H Grönberg and co-authors as published in Lancet Oncology (Nov. 9, 2015 http://dx.doi.org/10.1016/S1470-2045(15)00361-7) which is incorporated by reference herein. The protein biomarkers used in this study were measured using a multiplex platform based on ImmunoCAP ISAC technology (Thermo Fisher Scientific). In brief, approximately 70 patient samples, 12 calibrators and 12 control samples were measured using the same disposable test device. In a sensitivity analysis, each disposable device was given a multiplicative factor that was applied on all samples and all protein biomarkers on it. The multiplicative factors were allowed to vary within a defined span, and factors were chosen so as to increase the diagnostic performance in terms of ROC-AUC. When the multiplicative factors were allowed to vary between 0.96 and 1.04, i.e. when each disposable device was subjected to a systematic provocation of at the most 4%, the performance of the diagnostic assay was not improved in any significant manner. When the multiplicative factors were allowed to vary between 0.86 and 1.16, i.e. when each disposable device was subjected to a systematic provocation of at the most 16%, the performance of the diagnostic assay could be clearly improved from 0.74 to approximately 0.78. Such an increase in performance is larger than the contribution of any of the individual biomarkers except for total PSA. The conclusion of the sensitivity analysis is that the method disclosed by Gronberg and co-authors requires a high level of control so that systematic errors and shifts in assay performance are below 16%.

In parallel with the study conducted by Gronberg and co-authors, the performance of the total PSA assay on a Roche Cobas instrument in a regular clinical laboratory in Stockholm was assessed. Approximately 2 years of control sample data indicated that upon changing batch of calibration reagents, total PSA value of a control sample could shift in the order of 3-6%. Such a shift alone will not have a major impact on the performance of the screening method disclosed by Gronberg, but may become a concern when combined with other potential sources of systematic error.

Another activity conducted in parallel with the study conducted by Gronberg and co-authors was comparison of the total PSA value and the free PSA value from two independent instrument suppliers, Roche Cobas analyzer and BRAHMS Kryptor analyzer (Thermo Fisher Scientific) across 282 blood samples. Even though both platforms are approved for use in regular clinical laboratories, there was a systematic difference in reported values: total PSA from Kryptor assay was 5% greater than from Cobas assay, and free PSA from Kryptor assay was 16% greater than from Cobas assay.

Taken together, this illustrates that the multiplex assay disclosed by Gronberg will require strict control of systematic error, and show that there is a need for the present invention so as to make the method of Gronberg applicable in a wide spread manner. This is in line with the findings in the report “Between-method differences in prostate-specific antigen assays affect prostate cancer risk prediction by nomograms.” by C Stephan and co-authors as published in Clin Chem. 2011 July; 57(7):995-1004. doi: 10.1373/clinchem.2010.151472 as discussed elsewhere in the present disclosure.

From a general standpoint, multiplexed assays face many challenges to become reliably functional in a diagnostic or prognostic setting. The feature of multiplexing provides a different characteristic of data compared to singleplex assays, which in turn requires a different mindset when utilizing multiplexed data. Results of multiplexed data may preferentially be seen as a profile (a pattern, a fingerprint). A profile constitutes multiple data points that may have both relative and absolute relationships to each other. When comparing patterns, similarity can be claimed even if a fraction of the data points deviates from each other. This means that use of a pattern comprising multiple data points will be inherently robust to individual variations of the measured entities, and allows the multiplexed assay to have greater noise than what is typically acceptable for a singleplex assay. The redundancy property of multiplexed has been discussed in the context of prostate cancer diagnostics in WO 2014079865, which is incorporated by reference herein. Another generic issue for assays is systematic deviations. Systematic difference exist between instrument platforms, sometimes between disposable items used in the assay, and may also be caused by changes in the preanalytical procedures before running the assay. Systematic errors are complicating because they relate to the cut-off problem; at what level (magnitude or similarity) should a result be flagged as “positive”. In the case where a multiplexed assay is used for a diagnostic purpose, and where for some reason half of the data-points in the assay are shifted upwards 20%, results that were previously similar to a predetermined profile may not be that any more, resulting in inaccurate performance of the assay. The present invention addresses the systematic error by embedding a harmonization standard and transforming multiplex data to generalized units based on the harmonization standard result. Taken together, use of profiles while regarding the redundancy property disclosed in WO 2014079865 and implementing the harmonization standard procedure of the present invention, results in more reliable multiplexed assays.

### EXAMPLE 1

A subset of the data produced within the STHLM3 study was used in this example. STHLM3 is described in “Prostate cancer screening in men aged 50-69 years (STHLM3): a prospective population-based diagnostic study” by Gronberg and co-authors as published in Lancet Oncology (Volume 16, No. 16, p1667-1676, December 2015), which is incorporated by reference herein. The subset of data used in this example comprised protein biomarker data from 4335 individuals (3585 with Gleason Score below 7, 617 with Gleason Score 7, 133 with Gleason Score 8 or higher), more in particular blood concentration of total PSA, free PSA, intact free PSA, hK2, GDF-15 and MSMB. Blood concentration determinations were conducted using a microarray format where different antibodies were attached onto a substrate, the patient sample was allowed to contact the substrate, followed by contacting a set of conjugate molecules with the substrate, said set of conjugates containing molecules binding specifically to the different proteins captured from the patient sample onto the substrate. Conjugate molecules were labeled with a fluorescent dye so as to be quantifiable.

Each substrate had 96 independent and identical wells, meaning that 96 independent samples could be processed at the same time. Data for the current example was generated using 12 wells for calibration purposes, 9 wells for assay controls (three controls, each assayed in three independent wells), and three wells for a harmonization standard (one standard assayed in three independent wells). The remaining 72 wells were used for patient samples. The data generated for the current example hence consumed approximately 100 substrates, and the patient samples were distributed randomly across the substrates.

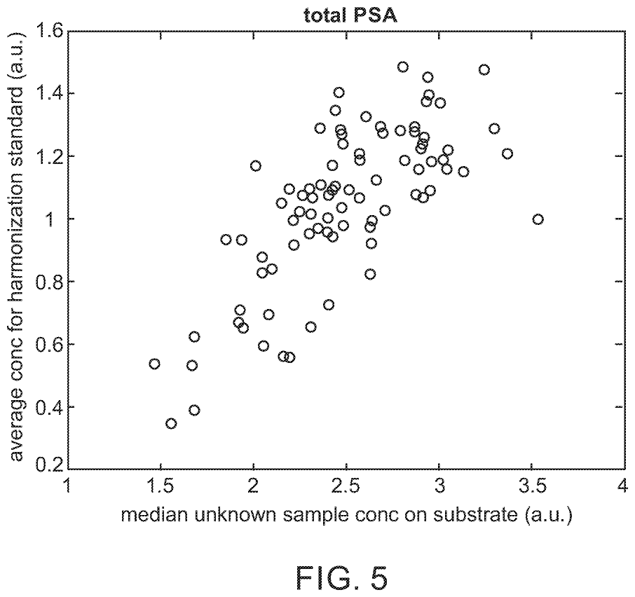

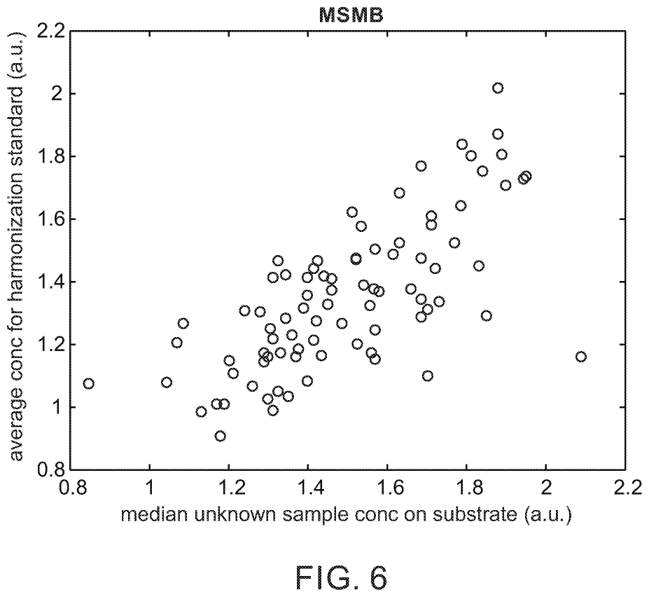

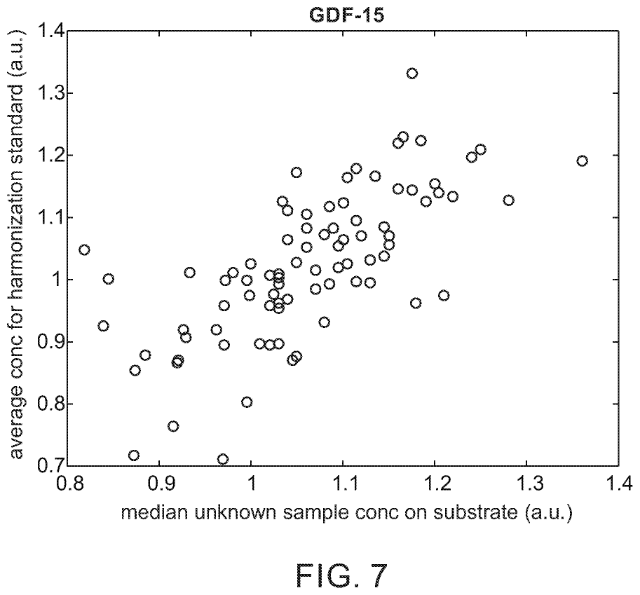

When comparing the average value for the harmonization standard with the median value of the 72 unknown samples within each substrate, three of the six protein biomarkers showed a correlation. FIG. 5 show the average total PSA value for harmonization standards (in arbitrary units) on the y-axis and the median unknown sample (i.e. patient sample) total PSA value on the same substrate on the x-axis (in the same arbitrary units). In cases where the harmonization standard results in a lower than average value, the median of the unknown samples was consistently below average. The results for MSMB and GDF-15 were similar, as shown in FIG. 6 and FIG. 7.

When comparing the 13 substrates (453 patients, of them 69 with Gleason Score 7 and 12 with Gleason Score 8 or higher) that had the lowest total PSA values for the harmonization standard to the remaining data set, median biomarker concentrations for total PSA and MSMB as presented in Table A were obtained. Median PSA values for patients assayed on the 13 selected substrates were, irrespective of the aggressiveness of cancer, consistently lower than the median value for benign non-cancer patients in the remaining data set. In addition, MSMB values for the selected 13 substrates were consistently higher, irrespective of aggressiveness of cancer, as compared to the remaining data set. Since total PSA is positively related to prostate cancer risk (i.e. higher value is indicative of higher risk) and MSMB is negatively correlated (i.e. a lower value is indicative of increased cancer risk, the 13 selected substrates had deviating values for both total PSA and MSMB, and simultaneously deviating in a manner that inaccurately mask risk.

TABLE A

| | Selected 13 substrates | | Remaining data set | |
|---|---|---|---|---|
| | Median total | Median total | Median total | Median total |
| | PSA for | MSMB for | PSA for | MSMB for |
| | unknown | unknown | unknown | unknown |
| | samples | samples | samples | samples |
| Benign | 1.91 | 1.62 | 2.51 | 1.43 |
| Gleason | 1.86 | 1.48 | 2.48 | 1.35 |
| Score = 6 | | | | |
| Gleason | 2.22 | 1.62 | 2.87 | 1.30 |
| Score = 7 | | | | |
| Gleason | 2.34 | 1.61 | 4.16 | 1.43 |
| score > 7 | | | | |

It is possible to adjust for the systematic deviation of the 13 selected substrates by transforming the concentration values produced by the assay through use of the values obtained for the harmonization standard samples on each substrate. Since the determination of concentration of any sample is associated with error, each substrate was transformed into generalized units using the following conservative transform: Correction factor=2/(1+(expected harmonization standard value−measured harmonization standard value)/(expected harmonization standard value)).

This transformation corrects only half of the deviation indicated by the harmonization standard, and it was selected to reduce potential measurement error on the harmonization sample to propagate into the generalized units. When comparing overall performance to discriminate aggressive cancer (defined as Gleason Score 7 or higher) from others, the use of generalized units did not improve the overall diagnostic performance from a population perspective. This means that (a) the vast majority of the produced data is good as is and (b) it is possible to implement generalized units in a safe manner from a population point of view. For the extreme cases, for example the 13 substrates discussed above, generalized units will be important and will improve accuracy for the patients involved. Hence, from an individual point of view, generalized units have a potential to correct assay results and produce adequate risk estimates. All in all, this suggest the present invention is of very high importance for individuals that are assayed on a substrate where harmonization samples indicate systematic deviation, and of moderate importance for the majority of individuals that are assayed on a substrate where harmonization samples are at normal levels.

Although the invention has been described with regard to its preferred embodiment, which constitutes the best mode currently known to the inventors, it should be understood that various changes and modifications as would be obvious to one having ordinary skill in this art may be made without departing from the scope of the invention as set forth in the claims appended hereto.

1. A method for harmonization of test results from a biological sample in a multiplexed biochemical assay, wherein a presence and/or concentration of multiple biomarkers are determined at the same time in the same sample and rendered comparable to test results obtained in different laboratories, said method comprising the steps a)-c),
a) quantifying (11) a presence and/or concentration of at least two different biomarkers (25, 26) in a biological sample and (27, 28) in a harmonization standard sample, independently in each sample, by means of a defined multiplexed biochemical assay device (20) implemented in a defined analytical instrument (22);
b) receiving (13) the test results (Test resultₛₐₘₚₗₑ, Test result_{harmonization}) from the samples into a computer-based decision engine (44); and
c) transforming (15) the test results (Test resultₛₐₘₚₗₑ) received from the biological sample, characterized in that transforming (15) the test results (Test resultₛₐₘₚₗₑ) from the biological sample includes transforming the test results (Test results_{harmonization}) from the harmonization standard sample into generalized units, GE, and adjusting (16) the results of the biological sample into the generalized units, GE.

2. The method according to claim 1, wherein the steps a) to c) are repeated at least twice within a particular time frame.

3. The method according to claim 2, wherein the steps a) to c) are repeated with a different biological sample and using the results (Test result_{harmonization}) from the at least two harmonization standards (HSS) quantified at different time points as input in the transformation (15) into generalized units (GE).

4. The method according to claim 2, wherein the steps a) to c) are repeated with a different biological sample (BS) and a different harmonization standard (HSS), and using the results (Test result_{harmonization}) from the at least two harmonization standards (HSS) quantified at different time points as input in the transformation (15) into generalized units (GE).

5. The method according to claim 2, wherein the steps a) to c) are repeated with a different biological sample (BS) or a different harmonization standard (HSS), and using the results (Test result_{harmonization}) from the at least two harmonization (HSS) standards quantified at different time points as input in the transformation (15) into generalized units (GE).

6. A defined multiplexed biochemical assay device (22) adapted to be implemented in a defined type of analytical instrument (34) and containing a presence or concentration of at least two different biomarkers (25, 26) in a biological sample (BS) and at least two different biomarkers (27, 28) in a harmonization standard sample (HSS), independently in each sample (BS, HSS), that can be quantified by the analytical instrument (34), characterized in that the biochemical assay device (22) comprises 10 independent and simultaneous assays, and wherein a harmonization standard (HSS) contains at least 90% of the biomarkers (27, 28) that are part of the assay.

7. The biochemical assay device according to claim 6, characterized in that at least two harmonization standards (HSS) are provided, said two harmonization standards (HSS) being designed to supply two different concentrations for at least 90% of the biomarkers (27, 28) that are part of the assay.

8. The biochemical assay device (22) according to claim 6, comprising a solid phase having immobilized thereon at least two different categories of ligands, wherein:
the first category of the ligands binds specifically to a PCa biomarker, and includes a plurality of different ligands binding specifically to each of a plurality of different PCa biomarkers, preferably at least one of PSA, iPSA, tPSA, fPSA, and hK2, and optionally MSMB and/or MIC-1; and
the second category of the ligands binds specifically to a SNPpc, and includes a plurality of different ligands binding specifically to each of a plurality of different SNPpc, such as at least one of rs11672691, rs11704416, rs3863641, rs12130132, rs4245739, rs3771570, rs7611694, rs1894292, rs6869841, rs2018334, rs16896742, rs2273669, rs1933488, rs11135910, rs3850699, rs11568818, rs1270884, rs8008270, rs4643253, rs684232, rs11650494, rs7241993, rs6062509, rs1041449, rs2405942, rs12621278, rs9364554, rs10486567, rs6465657, rs2928679, rs6983561, rs16901979, rs16902094, rs12418451, rs4430796, rs11649743, rs2735839, rs9623117, and rs138213197.

9. The biochemical assay device (22) according to claim 7, wherein the solid phase further has a third category of ligand immobilized which binds specifically to a SNPbm, and includes one or a plurality of different ligands binding specifically to one or each of a plurality of different SNPbm, such as at least one of rs1227732, rs3213764, rs1354774, rs2736098, rs401681, rs10788160, rs11067228, rs1363120, rs888663, and rs1054564.

10. A system (30) for harmonization of test results from a biological sample in a multiplexed biochemical assay, the system (30) comprising:
a defined multiplexed biochemical assay device (22) implemented in a defined type of analytical instrument (34), both (22, 34) adapted to quantify a presence or concentration of at least two different biomarkers (25, 26) in a biological sample (BS) and at least two different biomarkers in (27, 28) a harmonization standard sample (HSS), independently in each sample (BS, HSS), a computer-based decision engine (44) comprising:
a component (46) for receiving (13) the test results (TestBS, Test HSS) from the samples (BS, HSS) into the computer-based decision engine (44); and
a component (48) for transforming (15) the test results (TestBS) received from the biological sample (BS), characterized in that the component (28) for transforming (15) the test results from the biological sample (BS) is arranged to transform the test results (Test HSS) from the harmonization standard sample (HSS) into generalized units, GE, and arranged to adjust the test results of the biological sample into the generalized units, GE.
